(19) United States
(12) Patent Application Publication
(10) Pub. No.: US 2020/0308248 A1
(43) Pub. Date: Oct. 1, 2020

(54) Chimeric Natural Killer Cell Receptors and Method of Using Thereof

(71) Applicants: ST Phi Therapeutics, Seattle, WA (US); Yizun Biotech (Shanghai) Co., Ltd, Shanghai (CN); Yizun Biopharm (Shanghai) Co., Ltd, Shanghai (CN)

(72) Inventors: Steven Lingfeng Liu, Seattle, WA (US); Wenting Zhong, Wuping City (CN)

(21) Appl. No.: 16/827,697

(22) Filed: Mar. 23, 2020

Related U.S. Application Data
(60) Provisional application No. 62/919,750, filed on Mar. 26, 2019.

(51) Int. Cl. C07K 14/705; C07K 14/715; C07K 16/30; C07K 16/08; C12N 15/85; A61K 35/17

(52) U.S. Cl. CPC C07K 14/7056; C07K 14/7158; C07K 16/30; C07K 16/08; C07K 2317/53; A61K 35/17; C07K 2319/03; C07K 2319/02; C12N 15/85

(57) ABSTRACT

The present invention concerns providing chimeric natural killer cell receptor (CNK) constructs, genetically engineered T cells expressing such constructs (CNK-T), genetically engineered natural killer cells expressing such constructs (CNK-NK), and the use of CNK-T and CNK-NK to treat a variety of disease states. Specifically, the CNK are designed to target any types of infected, transformed, autoreactive, senescent and stressed cells overexpressing NKG2D ligands. Compared with native T cells and native natural killer cells, the CNK-T and CNK-NK are shown to have improved sensitivity in initiating cytotoxicity against the tumor cells and viral cells in absence or presence of the second genetically modification. Moreover, by incorporating the CNK into the chimeric antigen receptor (CAR) system, the genetically engineered T cells expressing such constructs (CNK/CAR-T) display enhanced sensitivity and superior cytotoxicity against tumor cells. Therefore, such CNK-T and CNK/CAR-T could be applied to the cellular therapy to treat tumor, virus infected diseases and autoimmune diseases directly.

### CROSS REFERENCE TO RELATED APPLICATION

This application claims priority under 35 U.S.C. § 119(e) to U.S. Provisional Patent application, Ser. No. 62/919,750, filed Mar. 23, 2019 filed, the disclosures of which are incorporated by reference in its entirety.

### FIELD

The present invention concerns providing chimeric natural killer cell receptor (CNK) constructs, genetically engineered T cells expressing such constructs (CNK-T), genetically engineered natural killer cells expressing such constructs (CNK-NK), and the use of CNK-T and CNK-NK to treat a variety of disease states. Specifically, the CNK are designed to target any types of infected, transformed, autoreactive, senescent and stressed cells overexpressing NKG2D ligands. Compared with native T cells and native natural killer cells, the CNK-T and CNK-NK are shown to have improved sensitivity in initiating cytotoxicity against the tumor cells and viral cells in absence or presence of the second genetically modification. Moreover, by incorporating the CNK into the chimeric antigen receptor (CAR) system, the genetically engineered T cells expressing such constructs (CNK/CAR-T) display enhanced sensitivity and superior cytotoxicity against tumor cells. Therefore, such CNK-T and CNK/CAR-T could be applied to the cellular therapy to treat tumor, virus infected diseases and autoimmune diseases directly.

### BACKGROUND

The ability of natural killer (NK) cells recognizing and killing tumor cells is based on NK activation associated with a key receptor, natural killer group 2 member D (NKG2D), a type II transmembrane-anchored C-type lectin-like protein. NKG2D belongs to the CD94/NKG2 family of C-type lectin-like receptors (Houchins et al., 1991), and is an activating/co-stimulatory immune receptor which regulates both innate and adoptive immune responses. In human, NKG2D is normally expressed in all NK, natural killer T cells (NKT), CD8+ T cells, and subsets of γδ T cells (Zhang et al., 2015). By binding ligands present in target cells, natural killer T cells (NKT) are activated and kill the targeted cells as an important modularity of the immune response.

In addition, there is growing evidence that NK cell-mediated immunoregulation plays an important role in the control of autoimmunity. Numerous studies suggested the involvement of NK cells in pathogenesis of such a common autoimmune diseases as juvenile rheumatoid arthritis, type I diabetes and autoimmune thyroid diseases. The defects of NK cells regulatory function as well as cytotoxic abilities are common in patients with autoimmune diseases with serious consequences including HLH hemophagocytic lymphocytosis (HLH) and macrophage activation syndrome (MAS) (Popko K et al., 2015).

NKG2D recognizes a broad and structurally diverse range of ligands with differential binding affinities (Champsaur and Lanier, 2010; Le Bert and Gasser, 2014). Many of these ligands are present in tumor cells, making NKG2D or its derivatives a potential powerful tool in eliminating these tumor cells and activate the immune system if proper activation of the NKG2D can be achieved once the binding of NKG2D to the ligands occurs. Human NKG2D recognizes family of MHC I Chain-related molecules A and B (MICA and MICB, generally termed as MIC) and family of six cytomegalovirus UL16-binding proteins (ULBP1-6), which are widely expressed in virus infected and malignant transformed cells but either absent or poorly expressed in normal healthy cells (Eagle and Trowsdale, 2007; El-Gazzar et al., 2013). Many primary tumor isolates from carcinoma (lung, breast, kidney, prostate, ovary, and colon), melanoma express MICA (Groh et al., 1999; Vetter et al., 2002). About 75% of primary cutaneous melanoma isolates and 50% of metastatic melanoma lesions express MICA protein (Vetter et al., 2002). In addition, human UL16-binding proteins (ULBP1-6) proteins are expressed by primary leukemia, glioma, and melanoma tumor cells (Friese et al., 2003; Pende et al., 2002; Salih et al., 2003). Moreover, human myeloma cells and over 80% of primary ovarian carcinoma cells express NKG2D ligands (Carbone et al., 2005; Carlsten et al., 2007). In addition, tumor cells extracted from patients with different types of leukemia, including AML (acute myeloid leukemia), ALL (acute lymphatic leukemia), CML (chronic myeloid leukemia), and CLL (chronic lymphatic leukemia), express heterogeneous levels of NKG2D ligands, MICA being the most highly expressed of all (Salih et al., 2003). Therefore, NKG2D ligands have been considered to be the promising targets for immunotherapy against virus infection and various types of cancer.

It has been reported that, after binding to its ligand, the NKG2D activation of natural killer (NK) cells involves a signaling pathway that requires recruitment of other molecules to the vicinity of NKG2D. NKG2D lacks a signaling motif in its cytoplasmic domain. Its signal transduction occurs upon ligation via the adaptor molecule to initiate signaling transduction and cellular activation. DNAX-activating proteins of 10 kDa (DAP10) was the first adaptor molecules identified to associate with NKG2D (Wu et al., 1999). The activated NKG2D in the cell membrane are composed of one NKG2D homodimer assembled with two DAP10 homodimers into a hexameric structure (Garrity et al., 2005). NKG2D also associates with DAP12, another DNAX-activating protein (Lanier et al., 1998).

The selection of adaptor molecule association depends on the cell types and the isoforms of NKG2D. In resting mouse NK cells, NKG2D exclusively associates with DAP10; whereas in activated NK, NKG2D associates with DAP10 and DAP12 (Diefenbach et al., 2002; Lanier et al., 1998). In human T cells, NKG2D exclusively associates with DAP10, due to structural differences in the transmembrane of mouse and human NKG2D and the lack of DAP12 in human T cells (Rosen et al., 2004). DAP10 and DAP12 association activates different signaling pathways. The DAP10 molecule contains an YXXM tyrosine-based motif that recruits the p85 subunit of phosphoinositidekinase-3 (P13K) and growth factor receptor-bound protein2(Grb2)(Wu et al., 1999). This signaling cascade is similar to which delivered by the T cell co-stimulatory molecules CD28 and ICOS. Therefore, during the NK activation, DAP10 could recruits downstream signaling effector molecules and enhance TCR-mediated signaling events, ultimately, results in cytotoxicity (Wallin et al., 2001). The DAP12 molecule contains an Immunoreceptor tyrosine-based activation motif (ITAM) which recruits ZAP70 (zeta-chain-associated protein kinase 70) and Syk (spleen tyrosine kinase) to directly mediate NK activation, cytokine production and cytotoxicity (Diefenbach et al., 2002). The difference of adaptor molecule association and the activation of signaling pathways may explain the functional differences of NKG2D in NK and T cells. In certain types of cells, binding of NKG2D to its ligands is sufficient to induce NK cell activation and cytotoxicity against NKG2D ligand expressed cells, but only co-stimulates CD8 T cells or γδT cells and synergy with TCR signaling (Groh et al., 2001; Wu et al., 2002).

However, it remains to be seen whether more effective and direct NK cell activation and cytotoxicity may be achieved by bringing together and physically tethering some components of the NKG2D signaling pathway. One potential advantage of tethering is to do away with the recruitment step and to produce a more effective activation Whether the potential advantage can be realized is not known and cannot be predicted by conjecturing, and simple experimentations cannot provide answers due to the myriad amounts of experimental designs. Thus it remains to be a highly desirable but unpredictable goal to transform native T cells to CNK-T in recognizing and killing the tumor cells via NKG2D signaling using a chimeric NK cell receptor that contains NKG2D ligand binding domain, an adaptor domain (chimeric DAP10/12), and an effector domain (such as CD3 zeta).

### SUMMARY

The present invention provides structures and compositions for chimeric NK receptor (CNK) constructs, T cells expressing the constructs (CNK-T), and methods of use of CNK-T and CNK/CAR-T. In some embodiments, the composition comprises a CNK receptor (CNK), wherein the CNK includes a full length or chimeric human NKG2D, with the adaptor protein DAP10 or/and DAP12, or DAP10/12 fusion directly fused to T cell activation signaling CD3ζ((CD3Z) or with the adaptor protein DAP10 fused to the cytoplasmic domain of DAP12, in presence or absence of the other stimulatory signaling moieties, such as CD28, 4-1BB (CD137), and OX40.

In some embodiments, an isolated chimeric NK receptor polypeptide (CNK) is disclosed, comprising an extracellular domain of human NKG2D comprising the polypeptide sequence of SEQ ID NO:12, and an adaptor protein binding to human NKG2D.

In some embodiments, the polypeptide of CNK is a full length human NKG2D comprising amino acid sequence of SEQ ID NO:1.

In some embodiments, the polypeptide further comprises a mouse NKG2D transmembrane comprising the amino acid sequence of SEQ ID NO:2, wherein the mouse NKG2D transmembrane is fused to the extracellular domain of human NKG2D.

In some embodiments, the adaptor protein is chosen from the group of DAP1 comprising the amino acid sequence of SEQ ID NO:3 and DAP12 comprising the amino acid sequence of SEQ ID NO:4.

In some embodiments, the adaptor protein is DAP10 or DAP12 fused to T cell activation signaling moiety with a flexible linker, wherein the flexible linker comprises from one to five tag cassettes, wherein each tag cassette is connected to one or more linker modules comprising a (Gly(x) Ser(y))n, wherein n is an integer from 1 to 10, and x and y are independently an integer from 0 to 10 provided that x and y are not both 0.

In some embodiments, the T cell activation signaling moiety is CD3Z comprising the amino acid sequence of Seq NO:5.

In some embodiments, The polypeptide further comprises the amino acid sequence of an effector domain, wherein the effector domain is a 4-1BB (CD137), CD2, CD3, CD35, CD25, CD27, CD28, CD30, CD40, CD79A, CD79B, CARD11, DAP10, DAP12, Fc receptor, Fyn, LIGHT, LTβR, HVEM, ICOS, Lck, LAG3, LAT, LRP, NOTCH1, NOTCH2, NOTCH3, NOTCH4, OX40 (CD134), ROR2, Ryk, SLAMF1, Slp76, pTa, TCRa, vTCRP, TRIM, Zap70, PTCH2, IL7 receptor, IL15 receptor, GITR or any combination thereof.

In some embodiments, the polypeptide sequence further comprises a chemokine receptor that helps to direct T cells moving toward chemokines expressed by tumors, the chemokine receptor is chosen from the group of CCR4, CCR5, CCR6, CCR7, CCR9, CCR2b, CXCR1, CXCR2, CXCR4.

In some embodiments, the polypeptide further comprises an engaging molecule.

In some embodiments, the engaging molecule is an artificial receptor that includes an extracellular ligand binding or affinity domain (AD), a hinge domain (HD), a transmembrane domain (TMD) and one or more intracellular domains (ICD).

In some embodiments, the ED is the single-chain variable fragment(scFv) or single-chain T cell receptor (scTCR) that specifically binds to a target antigen comprising as tumor associated antigens (TAA) or virus antigen (VA),

In some embodiments, the HD is selected from the hinge portion of IgG Fc fragment chosen from SEQ ID NO:6 and SEQ ID NO:7.

In some embodiments, the TMD is selected from DAP10 or DAP12 transmembrane domain, which comprises the amino acid sequence of SEQ ID NO:8 and SEQ ID N0:9, respectively.

In some embodiments, the polypeptide further comprises an ICD is selected from the intracellular domain (ICD) of the DAP10 or DAP12 fused to intracellular domain of T cell co-stimulatory molecules chosen from CD28, 4-1BB, OX40, and CD3Z.

In some embodiments, the engaging molecule comprises an affinity molecule chosen from scFv or single chain T cell receptor (scTCR), receptor ectodomain, and ligand binding molecules, which target TAA or VA.

In some embodiments, the polypeptide further comprises a Chimeric Antigen Receptor (CAR). The CAR comprises VH and VL portions of a scFv that targets TAA, a hinge chosen from a CD8a hinge or IgG4 hinge and configured to attach the scFv to a transmembrane domain, an intracellular effector comprising one or more co-stimulatory signaling domain comprising a CD28 intracellular domains(endodomain) or 4-1BB(CD137) intracellular domain, fused to CD3Z.

A method for using the polypeptide is provided, comprising the step of using a plasmid DNA, mRNA, lentiviral vector or retroviral vector that encodes the CNK sequence to transduce human T cells or NK cells to express CNK to treat viral diseases, cancer or autoimmune diseases.

In this method, human T cells are chosen from the group of CD3+ T cells, CD8+ T cells or CD4+ T cells isolated from the patients or healthy donor, and NK cells are chosen from donor NK or autologous NK cells.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF THE DRAWINGS

FIGS. 1A, 1B, 1C, 1D, 1E and 1F are schematic illustrations showing the constructed chimeric NK cell receptor (CNK) structural domains disposed in cell membranes, in according to some embodiments. FIG. 1A is a wide-type structure of human NKG2D (hNKG2D) (SEQ ID NO: 1), wherein the hNKG2D receptor forms a homodimer that associates with two homodimers of the adaptor molecules DAP10 (SEQ ID NO: 3) fused to the cytoplasmic domain of DAP12 (SEQ ID NO: 4); FIG. 1B shows a chimeric NKG2D receptor (SEQ ID NO: 2) is composed of human NKG2D extracellular domain (ED), mouse NKG2D transmembrane domain (TMD) and human NKG2D intracellular domain (ICD); the chimeric NKG2D receptor forms a homodimer that associates with two homodimers of the adaptor molecules DAP10 or DAP12; FIG. 1C shows the wide-type structure of human NKG2D, wherein the NKG2D receptor forms a homodimer that associates with two homodimers of the adaptor molecules DAP10 fused to the cytoplasmic domain of CD3Z (SEQ ID NO: 5); FIG. 1D shows the chimeric NKG2D receptor with mouse NKG2D transmembrane domain (TMD)) forming a homodimer that associates with two homodimers of the adaptor molecules DAP12 fused to the cytoplasmic domain of CD3Z; FIG. 1E shows the chimeric NK cell receptor in conjunction with a classic chimeric antigen receptor, which comprise a affinity moiety such as scFv or Fab antibody fragment, a hinge, the transmembrane domain, the co-stimulatory signaling domain of CD28 or 4-1BB. FIG. 1F shows the chimeric NK cell receptor in conjunction with a classic chimeric antigen receptor, which comprise a affinity moiety such as scFv or Fab antibody fragment, a hinge, the transmembrane domain, the co-stimulatory signaling domain of CD28 or 4-1BB, and the cytoplasmic signaling domain of CD3.

FIGS. 2A, 2B, 2c, 2D, 2E, and 2F are schematic diagrams showing the structure of the chimeric NK cell receptors, presented in correspondence to the chimeric NK cell receptors in FIGS. 2A-F. FIG. 2A: NKG2D-T2A-DAP10-DAP12-ICD; FIG. 2B: cNKG2D-T2A-DAP10-p2A-DAP12; FIG. 2C: NKG2D-T2A-DAP10-CD3Z; FIG. 2D: cNKG2D-T2A-DAP12-CD3Z; FIG. 2E: TAA scFv-CD28/41BB-T2A-NKG2D-T2A-DAP10-CD3Z; FIG. 2F: TAA scFv-CD28/41BB-Z-T2A-NKG2D-T2A-DAP10-CD3Z. (G4S: linkers (SEQ ID NO: 13); T2A belongs to 2A family of self-cleaving peptides (SEQ ID NO: 11); ECD: extra cellular domain; ICD: intracellular domain).

FIG. 3 is a series of flow cytometry profiles showing genetically modified T cells expressing NKG2D, i.e., expression of NKG2D in T cells transduced with NKG2D-T2A-DAP10-CD3Z. The native T cells express NKG2D only in CD8+ T cells; the transduced T cells express NKG2D in both CD8+ and CD4+ T cells

FIGS. 4A, 4B, and 4C are cytotoxicity assays examined under fluorescent and B/W microscopes showing cytotoxicity of NKG2D against HCC cell line HepG2 cells. FIG. 5A shows the phenotyping of Hepatocellular carcinoma (HCC) cell line HepG2 cells:CD3(−)CD45(−) MICA/B(+); FIG. 5B are optical microscopic images showing CNK-T are able to eradicate HepG2 after 24 h co-culture and form the proliferation clusters. Native T cells and CNK-T cells (NKG2D-T2A-DAP10-CD3Z) were co-culture with HepG2 with E:T ration 1:1 for 24 h; FIG. 5C are flow cytometry analysis of harvested cells post co-culture for 24 h. The data indicates CNK-T cells could significantly deplete HepG2 cells during co-culture and upregulate the expression for activation marker CD25.

FIGS. 5A and 5B are a series of flow cytometry profiles showing that strong cytotoxicity of NKG2D against acute myeloid leukemia (AML) cell line THP1 and MV411. FIG. 5A: Phenotyping of AML cell line: THP1 and MV411. Both cells expressing MICA/B and HLA-G; FIG. 5B: CNK-T cells efficiently eradicated AML cell lines, THP1 and MV411; Native T cells (NT) and CNK-T cells were co-cultured with THP1 or MV411 cells at the E:T=1:1; after 48 h, the cells were harvested and submitted to flow analysis. The data indicates CNK-T were able to significantly decrease the tumor cells and upregulate the expression for CD25.

FIG. 6 is a series of series of flow cytometry profiles showing CNK-T synergizes GPC3 CAR-T and increases cytotoxicity against HepG2 cells. Native T cells, GPC3 CAR-T cells and CNK/GPC3 CAR-T cells were co-culture with HepG2 cells at E:T ratio=1:5. 24 h post-culture, the cells were harvested and submitted to flow analysis. The data indicates the T cells co-expressing CNK and GPC3 CAR displayed superior cytotoxicity against HepG2 cells as compared to GPC3 CAR-T cells alone.

### DETAILED DESCRIPTION

In the following detailed description of embodiments of the invention, reference is made to the accompanying drawings in which like references indicates similar elements, and in which is shown by way of illustration, specific embodiments in which the invention may be practiced. These embodiments are described in sufficient detail to enable those skilled in the art to practice the invention. In other instances, well-known procedures, structures, and techniques have not been shown in detail in order not to obscure the understanding of this description. The following detailed description is, therefore, not to be taken in a limiting sense, and the scoop of the invention is defined only by the appended claims.

The present invention provides structures and compositions for chimeric NK receptor (CNK) constructs, T cells expressing the constructs (CNK-T), and methods of use of CNK-T and CNK/CAR-T. In some embodiments, the composition comprises a CNK receptor (CNK), wherein the CNK includes a full length or chimeric human NKG2D, with the adaptor protein DAP10 or/and DAP12, or DAP10/12 fusion directly fused to T cell activation signaling CD3ζ(or with the adaptor protein DAP10 fused to the cytoplasmic domain of DAP12, in presence or absence of the other stimulatory signaling moieties, such as CD28, 4-1BB (CD137), OX40.

In one embodiment, the CNK is introduced with other engaging molecule (EM) or Chimeric Antigen Receptor (CAR), which directs the CNK-T into the tumor antigen expression cells directly and promotes CNK-T proliferation. The EM could be the artificial receptor which is composed of tumor cells targeting antibody fragment or TCR, fused to cytoplasmic domain of co-stimulatory signaling. Moreover, the EM could be any affinity molecule or chemokine receptor, such as CCR5, CXCR4, which can further facilitate CNK-T to be recruited to the tumor site and display anti-tumor function. In some embodiment, the receptors might include the affinity moieties to tumor sites, such as the scFv target tumor antigen fused to IgG4 hinge, CD28 transmembrane, CD28 or 4-1BB intracellular domain. In some embodiment, the receptors might include the affinity moieties to tumor sites, such as the scFv target tumor antigen fused to IgG4 hinge, CD28 transmembrane, CD28 or 4-1BB intracellular domain and to the cytoplasmic domain of CD3((CD3Z).

The engagement element can be an antibody fragment with high affinity to a target antigen, or an extracellular domain of a receptor, in some embodiments. The engagement element can be an extracellular domain of a ligand, or a self-antigen, in some embodiments.

The 2A self-cleaving peptide is at equimolar levels of multiple genes on the same mRNA. T2A (SEQ ID NO:11) was used for the constructs.

In one embodiment, CNK is added with the chimeric antigen receptor (CAR), which is typically comprise an antibody moiety, preferably a scFv or Fab, attached via a linker to a transmembrane domain and two or more intracellular signaling domains, such as costimulatory signaling endodomain, such as CD28, ICOS, 4-1BB (CD137) alone or fused to CD3-z endodomain. Such design not only directs the CNK-T to the tumor sites more efficiently, but also synergizes and improves the CAR-T's cytotoxicity against tumor cells.

In some embodiments, CNK is introduced with engagement molecules, such as the tumor chemokine receptor, TAA (Tumor Associated Antigen) specific TCR, or TAA specific antibody fused to immune co-stimulatory domain, or TAA targeting chimeric antigen receptor (CAR). This allows the genetically engineered T cells migrate into the tumor sites and induce effective cytotoxicity against tumor cells.

In certain preferred embodiments, the target cell antigen may be Glypican-3 (GPC3), and the disease to be treated may be Hepatocellular carcinoma (HCC). In certain embodiments, the target cell antigen may be CD123, and the disease to be treated may be acute myeloid leukemia (AML).

HLA-G is a ligand for NK cell inhibitory receptor KIR2DL4, and therefore expression of this HLA-G by the tumor cells could defend against NK cell-mediated death and induce apoptosis of NK cells. In one embodiment, CNK-T cells don't express NK cell inhibitory receptor and resistance to HLA-G-mediated immune suppression and initiate cytotoxicity against the tumor cells.

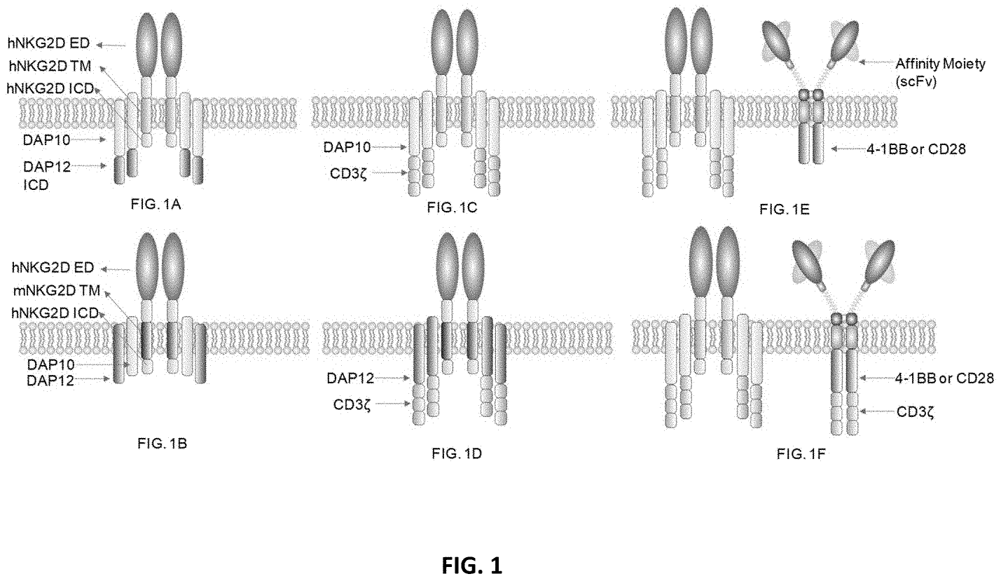

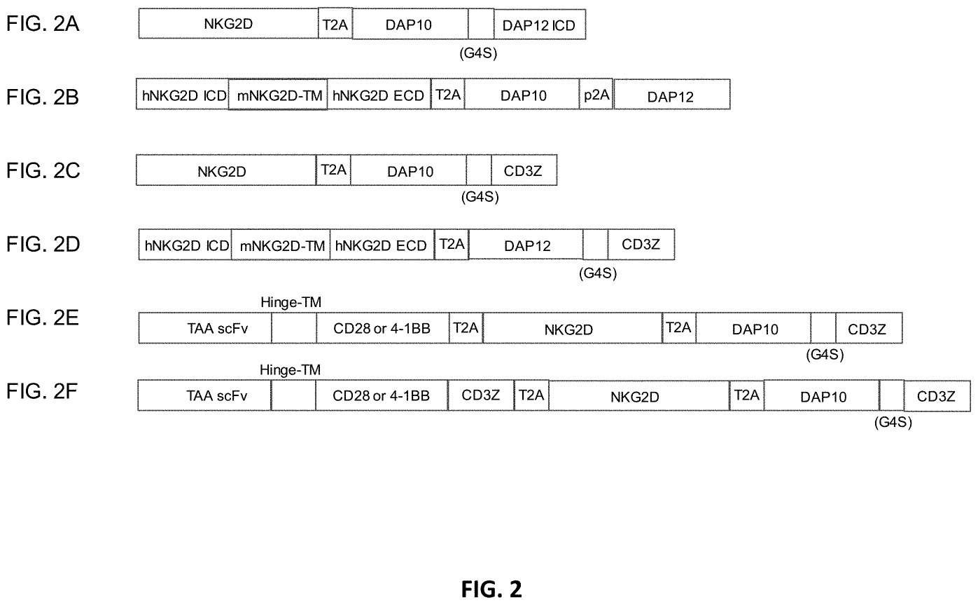

Examples of preferred embodiments of CNK are shown in FIG. 1 and FIG. 2. The chimeric human NKG2D is composed of human NKG2D extracellular domain, mouse NKG2D transmembrane domain and human NKG2D intracellular domain.

In one embodiment, the T cells or NK cells used to generate the CNK-T or CNK constructs are autologous cells obtained from the patient to be treated. More preferably, the T cells or NK cells used to generate the constructs are allogeneic cells.

Combined with the composition of the test device and the assembly diagram, the working principle and measuring method of the testing device is described in detail in the following. Reference is made to the accompanying drawings in which like references indicates similar elements, and in which is shown by way of illustration, specific embodiments in which the invention may be practiced. These embodiments are described in sufficient detail to enable those skilled in the art to practice the invention. In other instances, well-known circuits, structures, and techniques have not been shown in detail in order not to obscure the understanding of this description. The following detailed description is, therefore, not to be taken in a limiting sense, and the scoop of the invention is defined only by the appended claims.

In some embodiments, a chimeric natural killer receptor (CNK) is provided that comprises sequences of NKG2D and its adaptor proteins DAP10 or DAP12 fused to CD3Z (CD3ζ(eta), which enables the genetically engineered T cells to specifically recognize and eliminate the tumor cells or virus infected cells expressing NKG2D ligand.

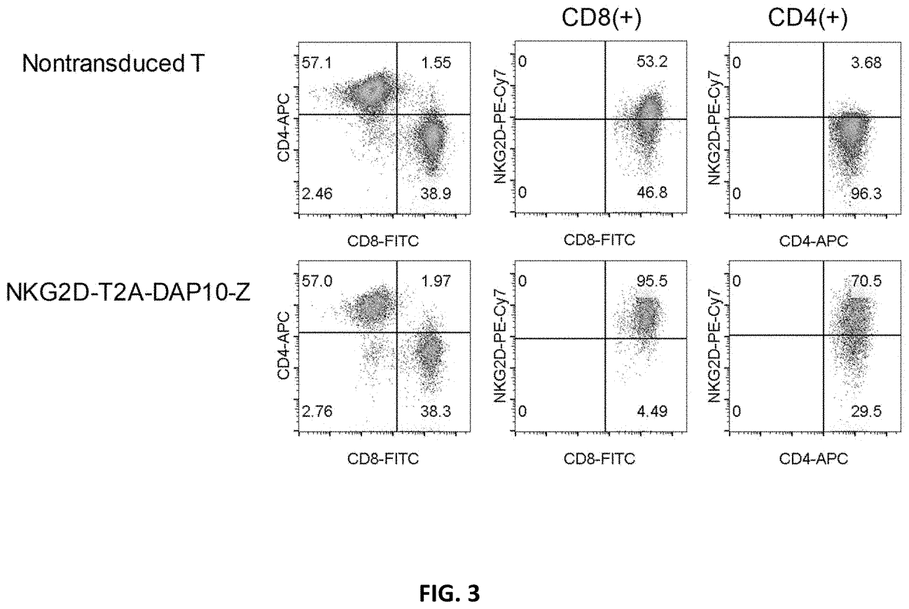

An exemplary lentiviral construct encoding a chimeric natural killer receptor comprising NKG2D-T2A-DAP10-CD3Z was designed, which comprises full-length human NKG2D, T2A self-cleavage peptide, the adaptor protein DAP10 fused to CD3 zeta chain. The DNA was synthesized and cloned into the lentiviral vector (such as pLenti CMV GFP-puro) and lentivirus were produced in 293T cells, using the package vectors (such as psPAX and pMD2G). The selected CD3+ T cells were stimulated by CD3/CD28 microbeads and infected by the lentiviral vector encoding NKG2D-T2A-DAP10-Z, and then expand in vitro for 12 days. The cells were submitted to flow cytometry to examine expression of NKG2D in CD8+ and CD4+ T cells (FIG. 3). The result indicated the transduced cells expressed high level of NKG2D on both CD8+ and CD4+ T cells, while for the nontransduced T cells, only CD8+ T cells expressed NKG2D.

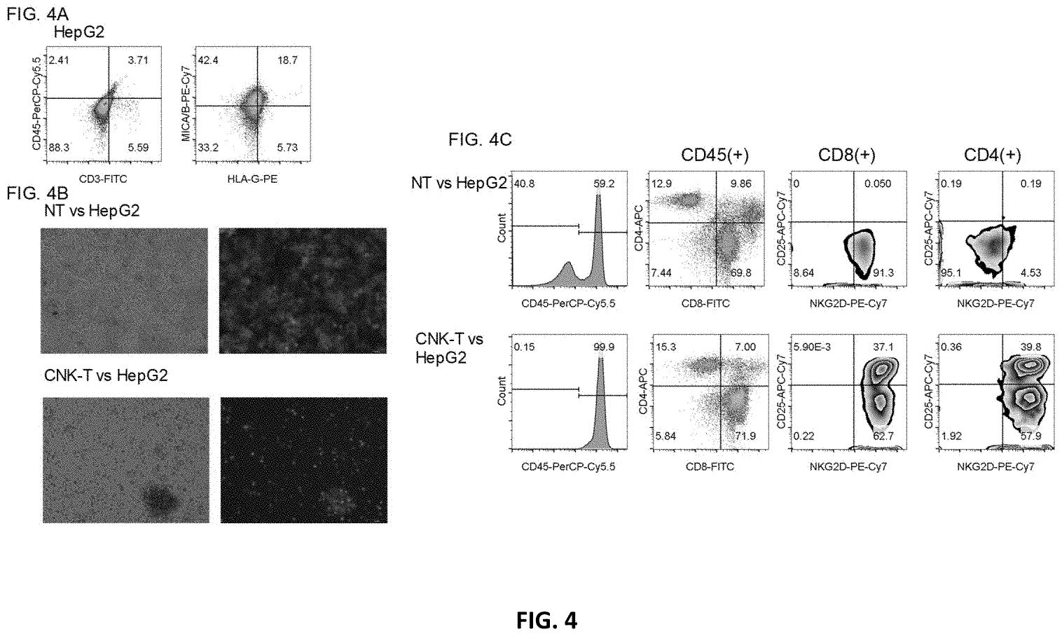

To examine CNK-T cells' cytotoxicity against tumor cells expressing NKG2D ligand, cytotoxicity was measured by testing whether lentivirus vector encoding NKG2D-T2A-DAP10-CD3Z transduced T cells (CNK-T) could eliminate the hepatocellular carcinoma (HCC) cell line HepG2 in vitro. As shown in FIG. 4A, HepG2 cells express NKG2D ligand, MICA/B, furthermore, HepG2 cells can be distinguished from T cells for their lack of expression of CD45 and CD3.

CNK-T were co-cultured with the HepG2 cells, at E:T ratio=1:10. After 48 h co-culture, the cells were observed under the fluorescence microscope to examine the CNK-T cells cytotoxicity against HepG2-EGFP cells. The result indicated CNK-T cells eradicated the HepG2 cells as efficiently compared to the non-transduced T cells (FIG. 4B).

To further examine the function of CNK design, the cells were also submitted to the flow cytometry to examine the efficiency of CNK-T cells' activation and cytotoxicity against the HepG2 cells. Since HepG2 cells were CD45 negative, it will be easy to distinguish the CD45+ T cells and HepG2 cells. As shown in FIG. 4C, the result indicated CNK-T cells were able to efficiently eradicate HepG2 cells in the co-culture according to the percentage of CD45(−) HepG2 cells left. Moreover, both CD8+ and CD4+ CNK-T significantly upregulated activation marker CD25 compared to the non-transduced T cells.

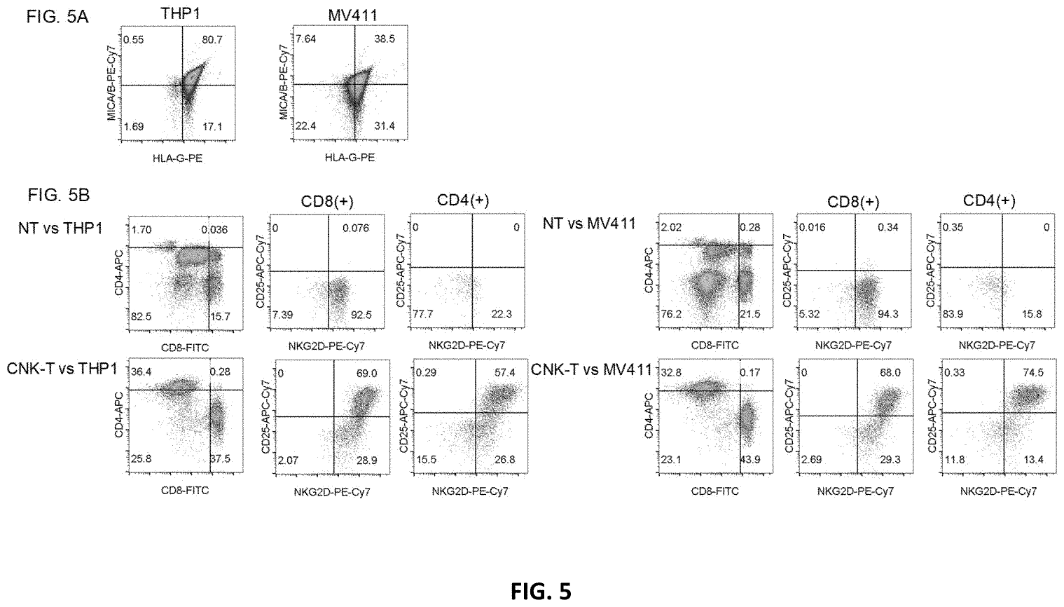

To further assess the cytotoxicity of CNK-T cells against other tumor cells, acute myeloid leukemia (AML) cell lines, THP1 and MV411, were examined to see whether lentivirus vector encoding NKG2D-T2A-DAP10-CD3Z transduced T cells (CNK-T) could eliminate the acute myeloid leukemia (AML) cell line, THP1 and MV411, which also express NKG2D ligand MICA/B (FIG. 5A).

CNK-T were co-cultured with the THP1 or MV411 cells, at E:T ratio=1:1. After 48 h co-culture, the cells were submitted to the flow cytometry to examine the cytotoxicity of CNK-T cells against the THP1 and MV411. Since T cells expresses high level of CD8 or CD4, it is feasible to distinguish the T cells and tumor cells. The result indicated CNK-T cells were able to efficiently eliminate both THP1 and MV411 cells in the co-culture according to the percentage of both CD8 and CD4 negative cells left. Moreover, both CD8+ and CD4+ CNK-T significantly upregulated activation marker CD25 and CD137 compared to the non-transduced T cells. Therefore, CNK-T cells can eliminate multiple tumor cells expressing NKG2D ligand.

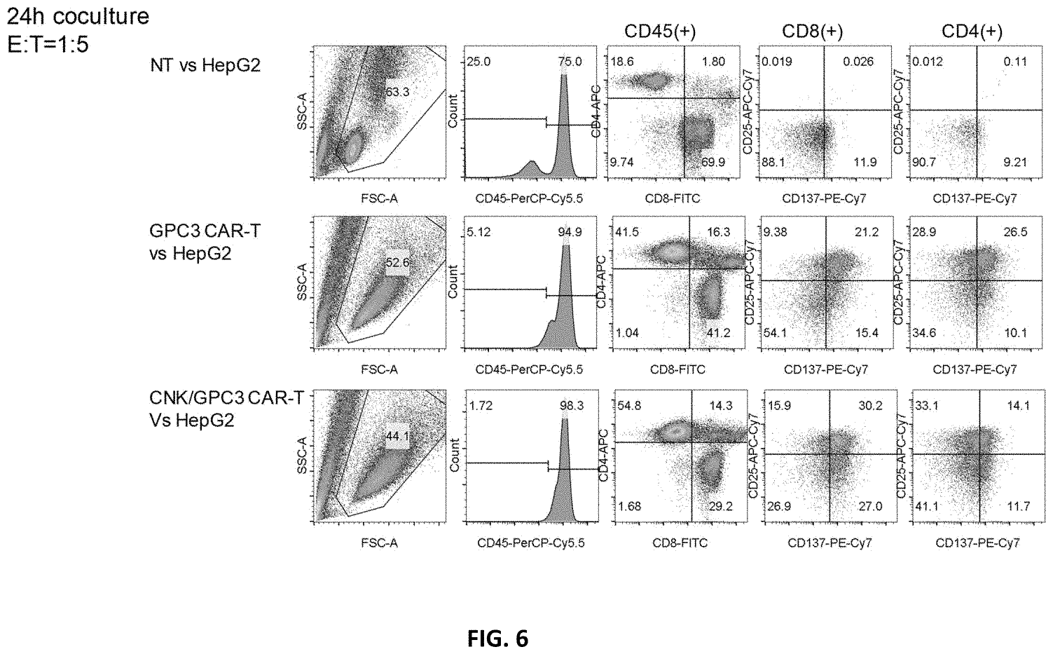

An exemplary lentiviral construct encoding a chimeric natural killer receptor comprising NKG2D-T2A-DAP10-CD3Z was introduced with anti-GPC3 CAR elements, which includes anti-GPC3 scFv (Nakano KYT et al., 2007), IgG4 hinge (SEQ ID NO: 7), CD28 transmembrane and cytoplasmic domain fused CD3 zeta chain (FIG. 1F and FIG. 6). The selected CD3+ T cells were stimulated by CD3/CD28 microbeads and infected by the lentivirus encoding NKG2D-T2A-DAP10Z-T2A-GPC3-CD28Z, and subsequently expanded in vitro for 12 days. To test whether inclusion of CNK in the CAR-T design improves the CAR-T cells' cytotoxicity against tumor cells, CNK/GPC3 CAR-T were co-cultured with the HepG2 cells transduced with EGFP, at E:T ratio=1:5. After 24 h co-culture, the cells were submitted to the flow cytometry to examine the cytotoxicity of CNK/GPC3 CAR-T against the HepG2 cells. The result indicated CNK/GPC3 CAR-T cells eradicate HepG2 cells more efficiently compared to the traditional GPC3 CAR-T cells in the co-culture according to the percentage of CD45(−) HepG2 cells left. Moreover, both CNK/GPC3 CAR-T and GPC3 CAR-T cells significantly upregulated activation marker CD25 and CD137 compared to the non-transduced T cells. Therefore, inclusion of CNK design does not interrupt the activation and cytotoxicity of anti-GPC3 CAR-T cells against the tumor cells. Moreover, the CNK design improves T cells function in eliminating tumor cells.

The target antigen can be a virus associated antigens (VA), which is selected from the any virus associated antigens, the exemplary virus antigen could be HPV associated antigen E6/E7, HBV antigen HBs Ag/HBe Ag, EBV antigens EBNA1/LMP1/LMP2/EBER, CMV antigen pp65/pp150/pp52/, HIV antigen p24, RSV, influenza A and B viruses, parainfluenza viruses, adenoviruses, coronavirus associated antigen S1/S2/N.

The target antigen can be a TAA, which is selected from the seven groups:

(1) Antigens Encoded by Mutated Genes, such as mutated CDK4, CTNNB1, CASP8, P53, KRAS, NRAS, EGFR, EGFRvIII, BRCA1, BRCA2, PALB2, ATM, RAD51D, RECQL, CHEK2, c-MET, or

(2) Cancer-Germline Genes, such as melanoma-antigen encoding (MAGE), MAGEA/MAGEB/MAGEC, BAGE, GAGE, LAGE/NY-ESO1, SSX genes, or

(3) Differentiation Genes derived from proteins that are expressed or overexpressed in a given type of tumor and the corresponding healthy tissue, such as tyrosinase, gp100/pmel17, Melan-A/MART-1, gp75/TRP1, TRP2, CEA, CLL1, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66ae, CD67, CD70, CD70L, CD74, CD79a, CD79b, CD80, CD83, CD95, CD123, CD126, CD132, CD133, CD138, CD147, CD154, gp100, or

(4) Overexpressed Antigens contributing to tumor growth or metastasis, such as RAGE-1, PRAME, survivin, ERBB2 (HER2/NEU), protein Wilms tumor 1 (WT1), EpCAM, MUC1(CA15-3), MUC2, MUC3, MUC4, MUC6, MUC16, PMSA, Placental growth factor (PIGF), HIF-1α, EGP-1 (TROP-2), EGP-2, surviving, epidermal glycoprotein 1 (EGP-1, TROP2), EGP-2, FLT3, G250, folate receptor, GAGE, gp100, HLA-DR, CD317(HM1.24), HMGB-1, or

(5) Embryonic antigen or fetal antigen or stem cell marker, such as CEA(CEACAM-5), CEACAM-6, AFP, OCT4, CD133, CD90, CD13, c-MET, CDC27, or

(6) Tumor metastasis associated chemokine receptor: such as CXCR2, CXCR4, CXCR7, CCR5, CCR7, CCR9, CCR10, CX3CR1 (Lazennec G et al., 2010), or

(7) Immune suppressive checkpoint: PD-L1, VISTA, Siglec-15.

The viral diseases susceptible to the treatment by these reagents and methods includes diseases caused by Coronavirus, SARS, MERS, Ebola, Cytomegalovirus(CMV), Epstein-Barr Virus (EBV), Human Papilloma Virus(HPV), Human T-Lymphotropic Virus(HTLV), Cold viruses, Influenza, Measles, Mumps, Rubella, Polio, Echo, Coxsackie, Hepatitis A, Hepatitis B, Hepatitis C, Rotavirus, Herpes 1 and 2, Rabies, Yellow fever, Dengue fever et al.

Cancers susceptible to the treatment by these reagents and method include: B-lineage acute lymphoblastic leukemia, B-cell chronic lymphocytic leukemia and B-cell non-Hodgkin's lymphoma. In another embodiment, the cancer is selected from the group consisting of lung cancer, melanoma, breast cancer, prostate cancer, colon cancer, renal cell carcinoma, ovarian cancer, neuroblastoma, rhabdomyosarcoma, lymphoma, acute lymphoblastic leukemia, small cell lung carcinoma, Hodgkin's lymphoma, childhood acute lymphoblastic leukemia, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, pancreatic cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, retinoblastoma, acute lymphocytic leukemia, acute myelocytic leukemia, chronic leukemia, polycythemia vera, lymphoma, multiple myeloma, Waldenstrom's macroglobulinemia, and heavy chain disease. In another embodiment, the cancer is selected from the group consisting of T-cell ALL, B-cell ALL, osteosarcoma, prostate carcinoma, rhabdomyosarcoma, neuroblastoma, Ewing sarcoma, colon carcinoma, gastric carcinoma, lung squamous cell carcinoma, hepatoma, and breast carcinoma.

Autoimmune disease susceptible to the treatment by these reagents and method includes type 1 diabetes, rheumatoid arthritis, systemic lupus erythematosus, and inflammatory bowel disease et al.

While the invention has been particularly shown and described as referenced to the embodiments thereof, those skilled in the art will understand that the foregoing and other changes in form and detail may be made therein without departing from the spirit and scope of the invention.

### REFERENCE

Carbone, E., P. Neri, M. Mesuraca, M. T. Fulciniti, T. Otsuki, D. Pende, V. Groh, T. Spies, G. Pollio, D. Cosman, L. Catalano, P. Tassone, B. Rotoli, and S. Venuta, 2005, HLA class I, NKG2D, and natural cytotoxicity receptors regulate multiple myeloma cell recognition by natural killer cells: Blood, v. 105, p. 251-8.
Carlsten, M., N. Bjorkstrom, H. Norell, Y. Bryceson, T. van Hall, B. Baumann, M. Hanson, K. Schedvins, R. Kiessling, H. Ljunggren, and K. Malmberg, 2007, DNAX accessory molecule-1 mediated recognition of freshly isolated ovarian carcinoma by resting natural killer cells: Cancer Research, v. 67, p. 1317-1325.
Champsaur, M., and L. Lanier, 2010, Effect of NKG2D ligand expression on host immune responses: Immunological Reviews, v. 235, p. 267-285.
Diefenbach, A., E. Tomasello, M. Lucas, A. Jamieson, J. Hsia, E. Vivier, and D. Raulet, 2002, Selective associations with signaling proteins determine stimulatory versus costimulatory activity of NKG2D: Nature Immunology, v. 3, p. 1142-1149.
Eagle, R., and J. Trowsdale, 2007, Promiscuity and the single receptor: NKG2D: Nature Reviews Immunology, v. 7, p. 737-744.
El-Gazzar, A., V. Groh, and T. Spies, 2013, Immunobiology and Conflicting Roles of the Human NKG2D Lymphocyte Receptor and Its Ligands in Cancer: Journal of Immunology, v. 191, p. 1509-1515.
Friese, M., M. Platten, S. Lutz, U. Naumann, S. Aulwurm, F. Bischof, H. Buhring, J. Dichgans, H. Rammensee, A. Steinle, and M. Weller, 2003, MICA/NKG2D-mediated immunogene therapy of experimental gliomas: Cancer Research, v. 63, p. 8996-9006.
Garrity, D., M. Call, J. Feng, and K. Wucherpfennig, 2005, The activating NKG2D receptor assembles in the membrane with two signaling dimers into a hexameric structure: Proceedings of the National Academy of Sciences of the United States of America, v. 102, p. 7641-7646.
Gasser, S., S. Orsulic, E. Brown, and D. Raulet, 2005, The DNA damage pathway regulates innate immune system ligands of the NKG2D receptor: Nature, v. 436, p. 1186-1190.
Groh, V., S. Bahram, S. Bauer, A. Herman, M. Beauchamp, and T. Spies, 1996, Cell stress-regulated human major histocompatibility complex class I gene expressed in gastrointestinal epithelium: Proc Natl Acad Sci USA, v. 93, p. 12445-50.
Groh, V., R. Rhinehart, J. Randolph-Habecker, M. Topp, S. Riddell, and T. Spies, 2001, Costimulation of CD8 alpha beta T cells by NKG2D via engagement by MIC induced on viral cells: Nature Immunology, v. 2, p. 255-260.
Groh, V., R. Rhinehart, H. Secrist, S. Bauer, K. Grabstein, and T. Spies, 1999, Broad tumor-associated expression and recognition by tumor-derived gamma delta T cells of MICA and MICB: Proceedings of the National Academy of Sciences of the United States of America, v. 96, p. 6879-6884.
Houchins, J. P., T. Yabe, C. McSherry, and F. H. Bach, 1991, DNA sequence analysis of NKG2, a family of related cDNA clones encoding type II integral membrane proteins on human natural killer cells: J Exp Med, v. 173, p. 1017-20.
Lanier, L., B. Corliss, J. Wu, C. Leong, and J. Phillips, 1998, Immunoreceptor DAP12 bearing a tyrosine-based activation motif is involved in activating NK cells: Nature, v. 391, p. 703-707.
Lazennec, G., and A. Richmond, 2010, Chemokines and chemokine receptors: new insights into cancer-related inflammation: Trends Mol Med, v. 16, p. 133-44.
Le Bert, N., and S. Gasser, 2014, Advances in NKG2D ligand recognition and responses by NK cells: Immunology and Cell Biology, v. 92, p. 230-236.
Pende, D., P. Rivera, S. Marcenaro, C. Chang, R. Biassoni, R. Conte, M. Kubin, D. Cosman, S. Ferrone, L. Moretta, and A. Moretta, 2002, Major histocompatibility complex class I-related chain a and UL16-binding protein expression on tumor cell lines of different histotypes: Analysis of tumor susceptibility to NKG2D-dependent natural killer cell cytotoxicity: Cancer Research, v. 62, p. 6178-6186.
Rosen, D., M. Araki, J. Hamerman, T. Chen, T. Yamamura, and L. Lanier, 2004, A structural basis for the association of DAP12 with mouse, but not human, NKG2D: Journal of Immunology, v. 173, p. 2470-2478.
Salih, H., H. Antropius, F. Gieseke, S. Lutz, L. Kanz, H. Rammensee, and A. Steinle, 2003, Functional expression and release of ligands for the activating immunoreceptor NKG2D in leukemia: Blood, v. 102, p. 1389-1396.
Venkataraman, G., D. Suciu, V. Groh, J. Boss, and T. Spies, 2007, Promoter region architecture and transcriptional regulation of the genes for the MHC class I-related chain A and B Ligands of NKG2D: Journal of Immunology, v. 178, p. 961-969.
Vetter, C., V. Groh, P. Straten, T. Spies, E. Brocker, and J. Becker, 2002, Expression of stress-induced MHC class I related chain molecules on human melanoma: Journal of Investigative Dermatology, v. 118, p. 600-605.
Wallin, J., L. Liang, A. Bakardjiev, and W. Sha, 2001, Enhancement of CD8(+) T cell responses by ICOS/B7h costimulation: Journal of Immunology, v. 167, p. 132-139.
Wu, J., V. Groh, and T. Spies, 2002, T cell antigen receptor engagement and specificity in the recognition of stress-inducible MHC class I-related chains by human epithelial gamma delta T cells: Journal of Immunology, v. 169, p. 1236-1240.
Wu, J., Y. Song, A. Bakker, S. Bauer, T. Spies, L. Lanier, and J. Phillips, 1999, An activating immunoreceptor complex formed by NKG2D and DAP10: Science, v. 285, p. 730-732.
Zhang, J., F. Basher, and J. D. Wu, 2015, NKG2D Ligands in Tumor Immunity: Two Sides of a Coin: Front Immunol, v. 6, p. 97.
Popko K, Górska E. The role of natural killer cells in pathogenesis of autoimmune diseases. Cent Eur J Immunol. 2015; 40(4):470-6.

SEQUENCE LISTING

SEQ ID NO: 1
Human NKG2D
MGWIRGRRSRHSWEMSEFHNYNLDLKKSDFSTRWQKQRCPVVKSKCRENA
SPFFFCCFIAVAMGIRFIIMVTIWSAVFLNSLFNQEVQIPLTESYCGPCP
KNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYI
ENCSTPNTYICMCIRTV
SEQ ID NO: 2
chimeric human NKG2D with mouse NKG2D
transmembrane(hmcNKG2D)
MGWIRGRRSRHSWEMSEFHNYNLDLKKSDFSTRWQKQRCPVVKSKCRENA
SPMFVVRVLAIALAIRFTLNTLMWLAIFKETFQPVLFNQEVQIPLTESYC
GPCPKNWICYKNNCYQFFDESKNWYESQASCMSQNASLLKVYSKEDQDLL
KLVKSYHWMGLVHIPTNGSWQWEDGSILSPLLTIIEMQKGDCALYASSFK
GYIENCSTPNTYICMQRTV
SEQ ID NO: 3
DAP10
MGGLEPCSRLLLLPLLLAVSGLRPVQAQAQSDCSCSTVSPGVLAGIVMGD
LVLTVLIALAVYFLGRLVPRGRGAAEAATRKQRITETESPYQELQGQRSD
VYSDLNTQRPYYK
SEQ ID NO: 4
DAP12
MIHLGHILFLLLLPVAAAQTTPGERSSLPAFYPGTSGSCSGCGSLSLPLL
AGLVAADAVASLLIVGAVFLCARPRRSPAQDGKVYINMPGRG
SEQ ID NO: 5
CD3ζeta
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
SEQ ID NO: 6
IgG1 hinge
EPKSCDKTHTCPPCP
SEQ ID NO: 7
IgG4 hinge
ESKYGPPCPSCP
SEQ ID NO: 8
DAP10 transmembrane domain
GVLAGIVMGDLVLTVLIALAV
SEQ ID NO: 9
DAP12 transmembrane domain
LVAADAVASLLIVGAVF
SEQ ID NO: 10
DAP10-DAP12 transmembrane domain fusion
GVLAGIVMGDLVLTVLIALAVLVAADAVASLLIVGAVF
SEQ ID NO: 11
T2A
GSGEGRGSLLTCGDVEENPGP
SEQ ID NO: 12
Human NKG2D extracellular domain
MGWIRGRRSR HSWEMSEFHN YNLDLKKSDF STRWQKQRCP
VVKSKCRENA SP
SEQ ID NO: 13
Linker
GGGGS

What is claimed is:

1. An isolated chimeric NK receptor polypeptide (CNK) comprising an extracellular domain of human NKG2D comprising the polypeptide sequence of SEQ ID NO:12, and an adaptor protein binding to human NKG2D.

2. The polypeptide of claim 1, further comprising a full length human NKG2D comprising amino acid sequence of SEQ ID NO:1.

3. The polypeptide of claim 1, further comprising a mouse NKG2D transmembrane domain, and a human NKG2D intracellular domain, wherein the extracellular domain of human NKG2D, the mouse NKG2D transmembrane, and the human NKG2D intracellular domain comprises the amino acid sequence of SEQ ID NO:2.

4. The polypeptide of claim 1, wherein the adaptor protein is chosen from the group of DAP1 comprising the amino acid sequence of SEQ ID NO:3 and DAP12 comprising the amino acid sequence of SEQ ID NO:4.

5. The polypeptide of claim 1, wherein the adaptor protein is DAP10 or DAP12 fused to T cell activation signaling moiety with a flexible linker, wherein the flexible linker comprises from one to five tag cassettes, wherein each tag cassette is connected to one or more linker modules comprising a (Gly(x) Ser(y))n, wherein n is an integer from 1 to 10, and x and y are independently an integer from 0 to 10 provided that x and y are not both 0.

6. The polypeptide of claim 5, wherein the T cell activation signaling moiety is CD3Z comprising the amino acid sequence of Seq NO:5.

7. The polypeptide of claim 6 where the amino acid sequence further comprises an effector domain, wherein the effector domain is a 4-1BB (CD137), CD2, CD3, CD35, CD25, CD27, CD28, CD30, CD40, CD79A, CD79B, CARD11, DAP10, DAP12, Fc receptor, Fyn, LIGHT, LTβR, HVEM, ICOS, Lck, LAG3, LAT, LRP, NOTCH1, NOTCH2, NOTCH3, NOTCH4, OX40 (CD134), ROR2, Ryk, SLAMF1, Slp76, pTa, TCRa, vTCRP, TRIM, Zap70, PTCH2, IL7 receptor, IL15 receptor, GITR or any combination thereof.

8. The polypeptide of claim 1, where the polypeptide sequence further comprises a chemokine receptor that helps to direct T cells moving toward chemokines expressed by tumors.

9. The polypeptide of claim 8, wherein the chemokine receptor is chosen from the group of CCR4, CCR5, CCR6, CCR7, CCR9, CCR2b, CXCR1, CXCR2, CXCR4.

10. The polypeptide of claim 1 where the polypeptide sequence further comprises an engaging molecule.

11. The polypeptide of claim 10, wherein the engaging molecule is an artificial receptor that includes an extracellular ligand binding or affinity domain (AD), a hinge domain (HD), a transmembrane domain (TMD) and one or more intracellular domains (ICD).

12. The polypeptide of claim 11, wherein the ED is the single-chain variable fragment(scFv) or single-chain T cell receptor (scTCR) that specifically binds to a target antigen comprising as tumor associated antigens (TAA) or virus antigen (VA),

13. The polypeptide of claim 11, wherein the HD is selected from the hinge portion of IgG Fc fragment chosen from SEQ ID NO:6 and SEQ ID NO:7.

14. The polypeptide of claim 11, wherein the TMD is selected from DAP10 or DAP12 transmembrane domain, which comprises the amino acid sequence of SEQ ID NO:8 and SEQ ID NO:9, respectively.

15. The polypeptide of claim 11, further comprising an ICD is selected from the intracellular domain (ICD) of the DAP10 or DAP12 fused to intracellular domain of T cell co-stimulatory molecules chosen from CD28, 4-1BB, OX40, and CD3Z.

16. The polypeptide of claim 10, wherein the engaging molecule comprises an affinity molecule chosen from scFv or single chain T cell receptor (scTCR), receptor ectodomain, and ligand binding molecules, which target TAA or VA.

17. The polypeptide of claim 1 where amino acid the sequence further comprises a Chimeric Antigen Receptor (CAR),

18. The polypeptide of claim 17, wherein the CAR comprises VH and VL portions of a scFv that targets TAA, a hinge chosen from a CD8a hinge or IgG4 hinge and configured to attach the scFv to a transmembrane domain, an intracellular effector comprising one or more co-stimulatory signaling domain comprising a CD28 intracellular domains(endodomain) or 4-1BB(CD137) intracellular domain, fused to CD3Z.

19. A method for using the polypeptide of claim 1, comprising the step of using a plasmid DNA, mRNA, lentiviral vector or retroviral vector that encodes the CNK sequence to transduce human T cells or NK cells to express CNK to treat viral diseases, cancer or autoimmune diseases.

20. The method of claim 19, wherein human T cells are chosen from the group of CD3+ T cells, CD8+ T cells or CD4+ T cells isolated from the patients or healthy donor, and NK cells are chosen from donor NK or autologous NK cells.


SEQUENCE LISTING (13 sequences)

SEQ ID NO: 1 - protein, 216 aa, Artificial Sequence
  synthetic sequence
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 2 - protein, 220 aa, Artificial Sequence
  synthetic sequence
MGWIRGRRSRHSWEMSEFHNYNLDLKKSDFSTRWQKQRCPVVKSKCRENASPMFVVRVLAIALAIRFTLNTLMWLAIFKETFQPVLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 3 - protein, 113 aa, Artificial Sequence
  syntheti sequence
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 4 - protein, 92 aa, Artificial Sequence
  syntheti sequence
MIHLGHILFLLLLPVAAAQTTPGERSSLPAFYPGTSGSCSGCGSLSLPLLAGLVAADAVASLLIVGAVFLCARPRRSPAQDGKVYINMPGRG

SEQ ID NO: 5 - protein, 112 aa, Artificial Sequence
  syntheti sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 15 aa, Artificial Sequence
  syntheti sequence
EPKSCDKTHTCPPCP

SEQ ID NO: 7 - protein, 12 aa, Artificial Sequence
  syntheti sequence
ESKYGPPCPSCP

SEQ ID NO: 8 - protein, 22 aa, Artificial Sequence
  syntheti sequence
EGVLAGIVMGDLVLTVLIALAV

SEQ ID NO: 9 - protein, 17 aa, Artificial Sequence
  syntheti sequence
LVAADAVASLLIVGAVF

SEQ ID NO: 10 - protein, 38 aa, Artificial Sequence
  syntheti sequence
GVLAGIVMGDLVLTVLIALAVLVAADAVASLLIVGAVF

SEQ ID NO: 11 - protein, 21 aa, Artificial Sequence
  syntheti sequence
GSGEGRGSLLTCGDVEENPGP

SEQ ID NO: 12 - protein, 52 aa, Artificial Sequence
  synthetic sequence
MGWIRGRRSRHSWEMSEFHNYNLDLKKSDFSTRWQKQRCPVVKSKCRENASP

SEQ ID NO: 13 - protein, 5 aa, Artificial Sequence
  synthetic sequence
GGGGS